(19) United States
(12) Patent Application Publication
(10) Pub. No.: US 2026/0202078 A1
(43) Pub. Date: Jul. 16, 2026

(54) INFORMATION PROCESSING METHOD, RECORDING MEDIUM, AND CALCULATION METHOD

(71) Applicant: TOPOLOGIC INC., Tokyo (JP)

(72) Inventor: Arata TAKAHASHI, Tokyo (JP)

(21) Appl. No.: 19/133,161

(22) PCT Filed: Dec. 14, 2023
(86) PCT No.: PCT/JP2023/044749

§ 371 Date: May 27, 2025

(30) Foreign Application Priority Data
Dec. 14, 2022 (JP) 2022-199825

(51) Int. Cl. F24F 11/64; F24F 11/52; F24F 11/89; F24F 120/14

(52) U.S. Cl. CPC F24F 11/64; F24F 11/52; F24F 11/89; F24F 2120/14

(57) ABSTRACT

An information processing method, a recording medium, and a calculation method are provided that make it possible to evaluate level of comfort according to individual differences. The information processing method comprises an acquisition step, a calculation step, and an output step. In the acquisition step, heat balance information that relates to the heat balance of a user is acquired. The heat balance information includes the flow of heat in the user. In the calculation step, the level of comfort of the user is calculated on the basis of the heat balance information and first reference information. The first reference information indicates the relationship between the heat balance information and the level of comfort. In the output step, information relating to the level of comfort is outputted.

### CROSS REFERENCE TO RELATED APPLICATIONS

This application is a 371 U.S. National Phase of International Application No. PCT/JP 2023/044749, filed on Dec. 14, 2023, which claims priority to Japanese Patent Application No. 2022-199825, filed Dec. 14, 2022. The entire disclosures of the above applications are incorporated herein by reference.

### BACKGROUND

### Technical Field

The present disclosure relates to an information processing method, a storage medium, and a calculation method.

### Related Art

JPH05-306827A discloses an air conditioner.

This air conditioner includes a cooling and heating means for cooling or heating a room, a PMV value calculation means for sequentially obtaining the PMV value of a living area by calculation, a PMV value display means for displaying the PMV value or an index thereof, a control target value designation means for designating a control target value within a comfortable range of PMV, and a cooling and heating control means for inputting the PMV value and the control target value and controlling the cooling and heating means so that the PMV value is stabilized at the control target value.

However, the air conditioner disclosed in JPH05-306827A cannot sufficiently evaluate a comfort level in consideration of individual differences, resulting in reduced productivity and wasteful use of energy.

In view of the above circumstances, the present disclosure provides an information processing method, a storage medium and a calculation method, etc. that enable evaluation of the comfort level considering individual differences.

### SUMMARY

According to an aspect of the present disclosure, an information processing method is provided. The information processing method includes an acquisition step, a calculation step, and an output step. The acquisition step includes acquiring heat balance information that is information related to heat balance of a user. The heat balance information includes a heat flow of the user. The calculation step includes calculating a comfort level of the user based on the heat balance information and a first reference information. The first reference information is information indicating a relationship between the heat balance information and the comfort level. The output step includes outputting information related to the comfort level.

According to such an aspect, it is possible to perform evaluation of the comfort level considering individual differences.

### BRIEF DESCRIPTION OF THE DRAWINGS

FIG. 1 is a configuration diagram showing an information processing system 100.

FIG. 2 is a block diagram showing a hardware configuration of an information processing apparatus 200.

FIG. 3 is a block diagram showing a hardware configuration of a smartphone 300.

FIG. 4 is a block diagram showing a hardware configuration of an air conditioner 400.

FIG. 5 is a diagram showing a configuration of a heat flow sensor 500.

FIG. 6 is a block diagram showing functions realized by an information processing apparatus 200 (controller 210).

FIG. 7 is an activity diagram showing a flow of calculation processing executed using the information processing system 100.

FIG. 8 shows an example in which the heat flow sensor 500 is attached to a user 800.

FIG. 9 shows an example in which the heat flow sensor 500 is attached to a sofa 900.

FIG. 10 is an activity diagram for explaining Activity A130 in FIG. 7 in more detail.

FIG. 11A and FIG. 11B are diagrams showing usage of the heat flow sensor 500 when the heat flow sensor 500 is attached to the skin of the user 800.

### DETAILED DESCRIPTION

Hereinafter, an embodiment of the present disclosure will be described with reference to the drawings. Various features described in the embodiment below can be combined with each other.

A program for realizing a software in the present embodiment may be provided as a non-transitory computer readable storage medium, may be provided for download from an external server, or may be provided in such a manner that the program can be activated on an external computer to realize function thereof on a client terminal (so-called cloud computing).

A term “unit” in the present embodiment may include, for example, a combination of a hardware resource implemented as circuits in a broad sense and information processing of software that can be concretely realized by the hardware resource. Furthermore, various kinds of information are described in the present embodiment, and such information may be represented by, for example, physical values of signal values representing voltage and current, high and low signal values as a set of binary bits consisting of 0 or 1, or quantum superposition (so-called qubits), and communication and computation may be executed on a circuit in a broad sense.

The circuit in a broad sense is a circuit realized by at least properly combining a circuit, circuitry, a processor, a memory, and the like. In other words, a circuit includes an application specific integrated circuit (ASIC), a programmable logic device (e.g., simple programmable logic device (SPLD), a complex programmable logic device (CLPD), field programmable gate array (FPGA), and the like.

### 1. Hardware Configuration

Section 1 describes a hardware configuration according to the present embodiment.

### 1-1. Information Processing System 100

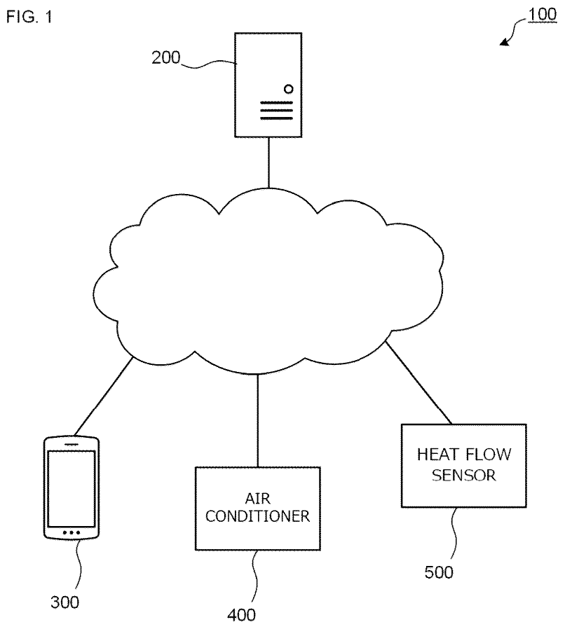

FIG. 1 is a configuration diagram showing an information processing system 100. The information processing system 100 includes an information processing apparatus 200, a smartphone 300, an air conditioner 400, a heat flow sensor 500, which are connected through a network. These components will be described in further detail below. Here, a system exemplified by the information processing system 100 is composed of one or more devices or components. Thus, for example, even the information processing apparatus 200 alone may be a system exemplified by the information processing system 100.

### 1-2. Information Processing Apparatus 200

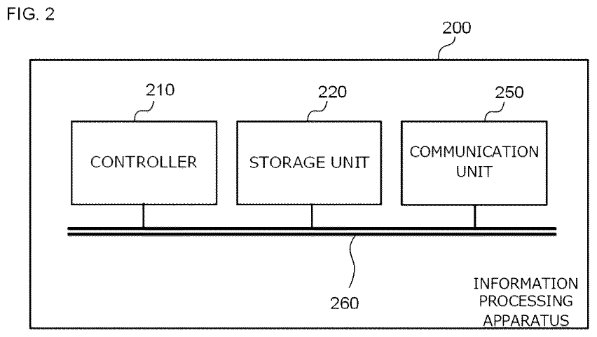

FIG. 2 is a block diagram showing a hardware configuration of the information processing apparatus 200. The information processing apparatus 200 includes a controller 210, a storage unit 220, and a communication unit 250, and these components are electrically connected via a communication bus 260 inside the information processing apparatus 200. Each of the components will be described in further detail.

The controller 210 processes and controls overall operation pertaining to the information processing apparatus 200. The controller 210 is, for example, an unshown CPU (Central Processing Unit). The controller 210 is configured to read a predetermined program stored in the storage unit 220 to realize various functions with respect to the information processing apparatus 200. In other words, information processing by software stored in the storage unit 220 can be executed as each function unit included in the controller 210 by specifically realized through the controller 210, that is an example of hardware. These will be described in further detail in Section 2. The controller 210 is not limited to being a single controller but may be implemented with a plurality of controllers 210 for each function. Moreover, a combination thereof may be applied.

The storage unit 220 is configured to store various information necessary for information processing of the information processing apparatus 200. This can be implemented, for example, as a storage device such as an SSD (Solid State Drive) that stores various programs, etc. related to the information processing apparatus 200 executed by the controller 210, or as a memory such as an RAM (Random Access Memory) that stores temporarily necessary information (arguments, sequences, etc.) in relation to calculations of a program. Further, a combination thereof may be applied.

Although wired communication means such as USB, IEEE1394, Thunderbolt (registered trademark), wired LAN network communication, etc. are preferred, the communication unit 250 may include wireless LAN network communication, mobile communication such as 5G/LTE/3G, Bluetooth (registered trademark) communication, etc. as necessary. That is, it is further preferable to implement as a set of these communication means. In other words, the information processing apparatus 200 communicates various information with the smartphone 300 via the network through the communication unit 250.

### 1-3. Smartphone 300

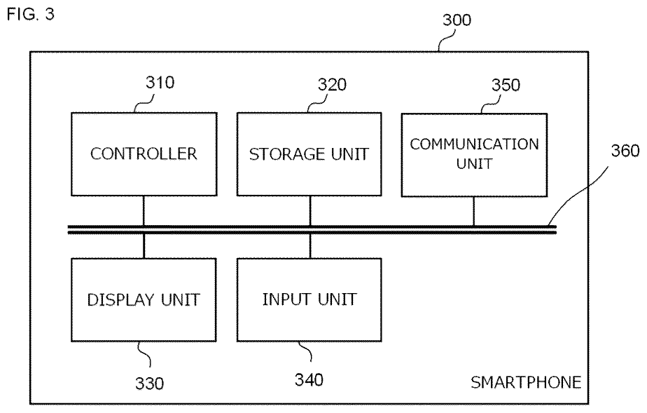

FIG. 3 is a block diagram showing a hardware configuration of the smartphone 300. The smartphone 300 includes a controller 310, a storage unit 320, a display unit 330, an input unit 340, and a communication unit 350. These components are electrically connected via a communication bus 360 inside the smartphone 300. Descriptions of the controller 310, the storage unit 320 and the communication unit 350 are omitted since they are substantially the same as those of the controller 210, the storage unit 220 and the communication unit 250 in the information processing apparatus 200.

The display unit 330 may be included in the housing of the smartphone 300 or may be attached thereto externally. The display unit 330 displays a screen of a graphical user interface (GUI) that can be operated by a user. For instance, this is preferable to be implemented by using different display devices such as a CRT display, a liquid crystal display, an organic EL display, and a plasma display, depending on the type of the smartphone 300. Hereinafter, the display unit 330 is described as being included in the housing of the smartphone 300.

The input unit 340 may be included in the housing of the smartphone 300 or may be attached thereto externally. For example, the input unit 340 may be integrated with the display unit 330 and implemented as a touch panel. If the input unit 340 is implemented as a touch panel, the user can perform the input by tap operations, swipes, etc. Needless to say, a switch button, a mouse, a QWERTY keyboard or the like may be employed instead of a touch panel. That is, the input unit 340 receives an operation input performed by the user. The input is transferred to the controller 310 via the communication bus 360 as an instruction signal. The controller 310 can then execute predetermined control or calculation as necessary.

### 1-4. Air Conditioner 400

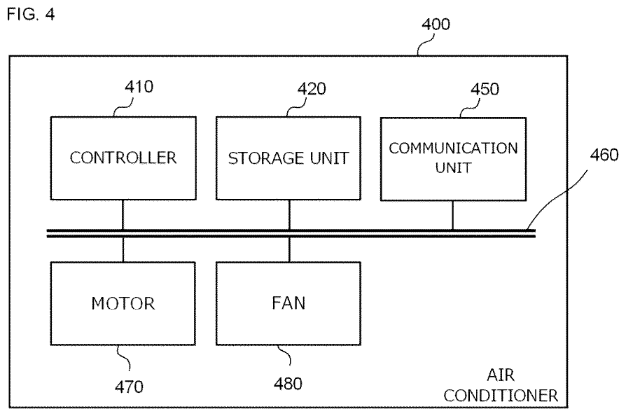

FIG. 4 is a block diagram showing a hardware configuration of an air conditioner 400. The air conditioner 400 includes a controller 410, a storage unit 420, a communication unit 450, a motor 470, and a fan 480, and these components are electrically connected inside the air conditioner 400 via a communication bus 460. Descriptions of the controller 410, the storage unit 420, and the communication unit 450 are omitted since they are substantially the same as those of the controller 210, the storage unit 220, and the communication unit 250 in the information processing apparatus 200.

The motor 470 serves as a drive source that generates the driving force. The motor 470 is arranged in an unshown case. The motor 470 is connected to the fan 480. The motor 470 may be, for example, an electric motor. The motor 470 generates driving force by receiving supply of electric power.

The fan 480 generates airflow by rotating. The fan 480 is arranged inside the same case as the motor 470. The fan 480 is connected to the motor 470. The fan 480 may be, for example, a sirocco fan. The fan 480 rotates by the driving force transmitted from the motor 470.

### 1-5. Heat Flow Sensor 500

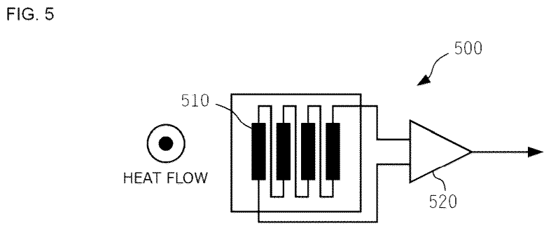

FIG. 5 is a diagram showing a configuration of the heat flow sensor 500. The heat flow sensor 500 is a sensor that detects the amount of transfer of heat energy. The heat flow sensor 500 usually detects the amount of transfer (heat flux) per unit area and is also called a heat flux sensor. The heat flow sensor 500 is not a sensor with a large heat capacity such as a pyroelectric sensor, but a thin-film type heat flow sensor is used as the heat flow sensor 500.

The heat flow sensor 500 is not particularly limited as long as it may be made of, for example, a topological material (a topological ferromagnetic material or a topological antiferromagnetic material), which is called Weyl semimetals, or it may be made of a material capable of sensing by thermoelectric conversion based on the anomalous Nernst effect such as ferrimagnetic materials, Heusler alloys, etc. Such a heat flow sensor 500 has a significantly lower heat capacity than the sensors based on the Seebeck effect, and thus has a very high responsiveness when the heat flows in or out.

The heat flow sensor 500 includes a thermoelectric conversion part 510 and an amplifier 520. The thermoelectric conversion part 510 is an element that performs thermoelectric conversion. The thermoelectric conversion part 510 outputs a signal generated by the transfer of heat energy (hereinafter referred to as a “heat flow sensor signal”) to the amplifier 520. The intensity of the heat flow sensor signal is expressed as the formula k*M*Q (k is a constant, M is the magnetization of the heat flow sensor, and Q is the heat flow). The amplifier 520 is a circuit that amplifies the current generated by the thermoelectric conversion part 510 through thermoelectric conversion. The amplifier 520 transmits the digitized heat flow sensor signal to the information processing apparatus 200 via a communication unit (not shown).

### 2. Functional Configuration

Section 2 describes a functional configuration of the present embodiment. As mentioned above, information processing by software stored in the storage unit 220 is specifically realized by the controller 210, which is an example of hardware, thereby being executed as each function unit included in the controller 210.

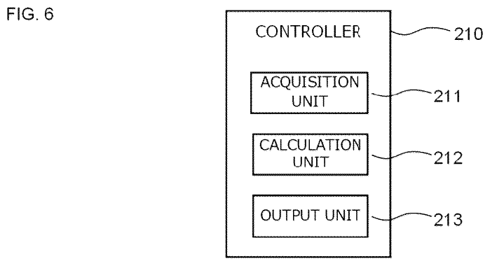

FIG. 6 is a block diagram showing functions realized by the information processing apparatus 200 (controller 210). The information processing apparatus 200 (controller 210) includes an acquisition unit 211, a calculation unit 212, and an output unit 213, which correspond respectively to the steps of the information processing method of the present embodiment.

The acquisition unit 211 is configured to acquire various types of information. The acquisition unit 211 is configured to execute an acquisition step. For example, the acquisition unit 211 acquires heat balance information that is information related to the heat balance of the user. Here, the heat balance information includes the heat flow of the user 800.

The calculation unit 212 is configured to calculate various types of information. The calculation unit 212 is configured to execute a calculation step. For example, the calculation unit 212 calculates a comfort level of the user 800 based on the acquired heat balance information and a first reference information. Here, the first reference information is information indicating a relationship between the heat balance information and the comfort level.

The output unit 213 is configured to output various types of information. The output unit 213 is configured to execute an output step. For example, the output unit 213 outputs information related to the calculated comfort level of the user 800.

### 3. Calculation Method

Section 3 describes a calculation method using the information processing system 100 described above. The calculation method is a method for calculating the comfort level of the user 800 by using the heat flow sensor 500. The calculation method includes an arrangement step, a measurement step, and a calculation step. In the arrangement step, the heat flow sensor 500 is arranged at a position where the heat flow of the user 800 can be measured. In the measurement step, the heat flow of the user 800 is measured by using the heat flow sensor 500. In the calculation step, the comfort level of the user 800 is calculated based on the heat flow of the user 800 and a third reference information. The third reference information is information indicating a relationship between the heat flow of a user and the comfort level of the user.

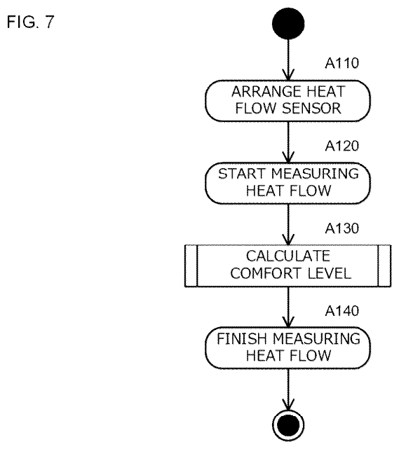

FIG. 7 is an activity diagram showing a flow of calculation processing executed using the information processing system 100. The following description follows each activity in this activity diagram.

First, the user 800 arranges the heat flow sensor 500 at a position where the heat flow of the user 800 can be measured (Activity A110). That is, the user 800 may, for example, attach the heat flow sensor 500 to the user 800, or more specifically, attach the heat flow sensor 500 to the skin of the user 800. Moreover, the user 800 may arrange the heat flow sensor 500 on a seat surface 910 of a sofa 900 on which the user 800 sits.

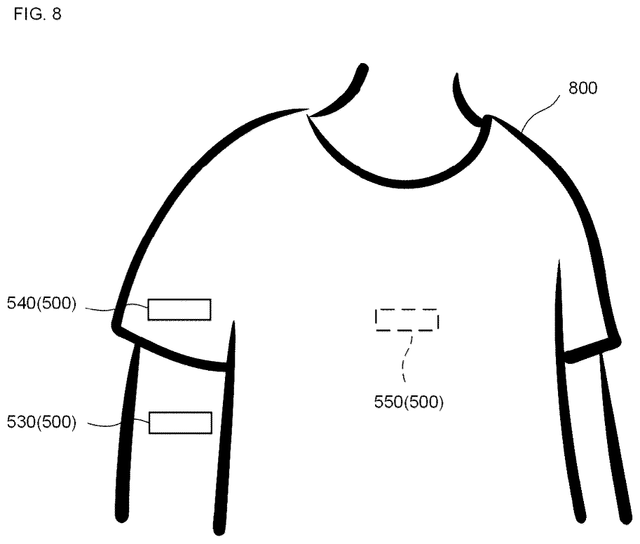

FIG. 8 shows an example in which the heat flow sensor 500 is attached to the user 800. A heat flow sensor 530 is attached in direct contact with the arm of the user 800. A heat flow sensor 540 is attached to the arm of the user 800 over his/her clothing. A heat flow sensor 550 is attached in direct contact with the chest of the user 800 under the clothing. In this way, the position at which the heat flow sensor 500 is attached to the user 800 is not particularly limited as long as it is a position at which the heat flow of the user 800 can be measured.

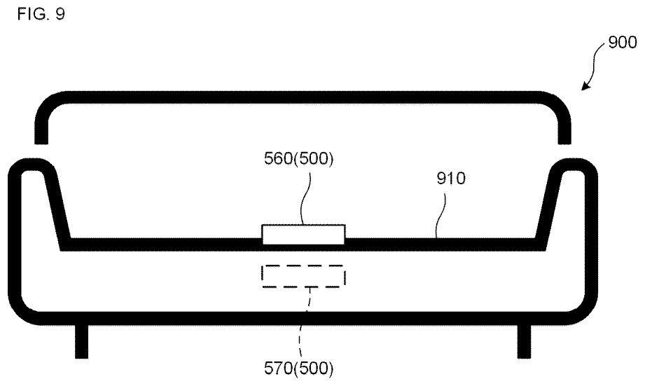

FIG. 9 shows an example in which the heat flow sensor 500 is attached to the sofa 900. A heat flow sensor 560 is attached to the seat surface 910 of the sofa 900. A heat flow sensor 570 is attached inside the seat surface 910 of the sofa 900. In this way, the position at which the heat flow sensor 500 is attached to the sofa 900 is not particularly limited as long as the heat flow of the user 800 can be measured.

To rephrase this activity into a step, in the arrangement step, the heat flow sensor 500 is arranged at a position where the heat flow of the user 800 can be measured. In the arrangement step, the heat flow sensor 500 may be attached to the user 800. In the arrangement step, the heat flow sensor 500 is preferably attached to the skin of the user 800. In the arrangement step, the heat flow sensor may be arranged on the seat surface 910 of the sofa 900 on which the user 800 sits. According to such an aspect, the heat flow of the user 800 can be acquired more reliably.

As shown in FIG. 8 and FIG. 9, in the arrangement step, a plurality of heat flow sensors 500 may be arranged at positions where the heat flow of the user 800 can be measured. In this case, in the measurement steps in activities A120 to A130 described below, the heat flows are measured from each of the heat flow sensors 500. By using a plurality of heat flow sensors 500, measurement errors that occur in individual heat flow sensors 500 can be corrected and reduced. That is, according to such an aspect, the influence of thermal resistance in the heat flow sensor 500 can be reduced.

Next, the heat flow sensor 500 starts measuring the heat flow of the user 800 (Activity A120). To rephrase this activity into a step, in the measurement step, the heat flow of the user 800 is measured by using the heat flow sensor 500.

The information processing apparatus 200 then calculates the comfort level of the user 800 (Activity A130). The comfort level of the user 800 may be indicated as a thermal index, and specifically, may be, for example, effective temperature (ET), operative temperature (OT), a wind chill index (WCI), a heat stress index (HSI), wet bulb globe temperature (WBGT), temperature-humidity index (THI), predicted mean vote (PMV), and standard new effective temperature (SET). To rephrase this activity into a step, in the calculation step, the comfort level of the user 800 is calculated based on the heat flow of the user 800 and a third reference information. The third reference information is information indicating a relationship between the heat flow of a user and the comfort level of the user. Activity A130 will be described later.

Next, the heat flow sensor 500 finishes measuring the heat flow of the user 800 (Activity A140). In Activity A140, the measurement of the heat flow of the user 800 may be terminated, for example, when the user 800 removes the heat flow sensor 500 from himself/herself.

According to the aspect of Section 3, it is possible to perform evaluation of the comfort level considering individual differences. In addition, due to its simple configuration, the saved resources can be used for other core functions.

### 4. Information Processing Method

Section 4 describes an information processing method that uses the above-mentioned information processing system 100. The information processing method includes an acquisition step, a calculation step, and an output step. In the acquisition step, heat balance information that is information related to the heat balance of the user 800 is acquired. The heat balance information includes the heat flow of the user 800. In the calculation step, the comfort level of the user 800 is calculated based on the heat balance information of the user 800 and a first reference information. The first reference information is information indicating a relationship between the heat balance information of a user and the comfort level of the user. In the output step, information related to the comfort level of the user 800 is output.

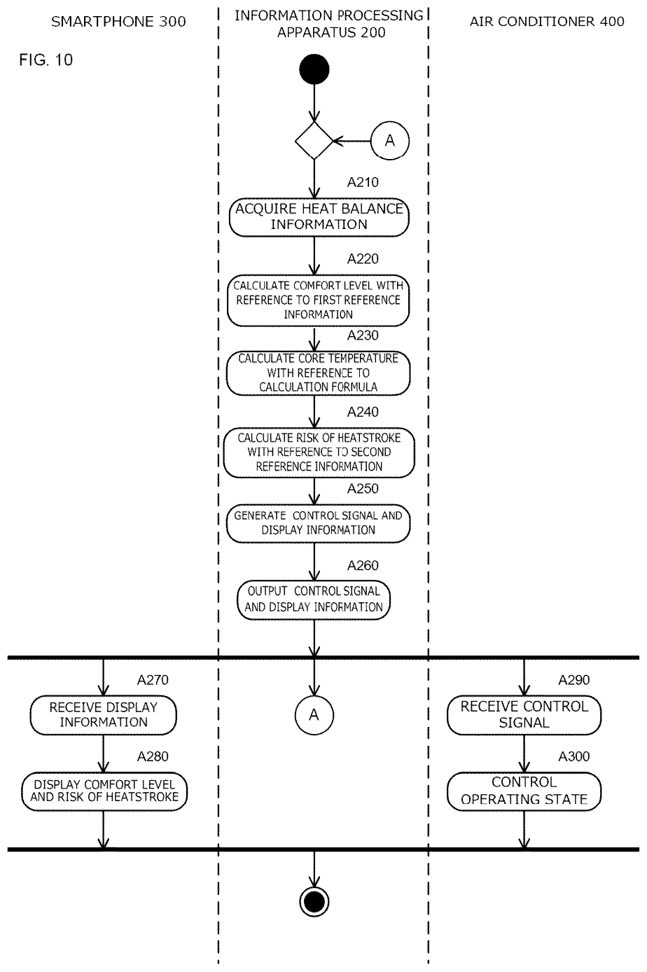

FIG. 10 is an activity diagram for explaining Activity A130 in FIG. 7 in more detail. The following description follows each activity in this activity diagram.

First, the controller 210 in the information processing apparatus 200 acquires heat balance information which is information related to the heat balance of the user 800, from the heat flow sensor 500 (Activity A210). The controller 210 acquires a heat flow sensor signal of the heat flow sensor 500 as heat balance information. To rephrase this activity into a step, in the acquisition step, heat balance information which is information related to the heat balance of the user 800 is acquired. Here, the heat balance information includes the heat flow of the user 800.

In Activity A210, for example, the following three-step information processing is executed. (1) The communication unit 250 receives a heat flow sensor signal transmitted from the heat flow sensor 500. (2) The controller 210 acquires the heat flow sensor signal via the communication bus 260. (3) The controller 210 causes the storage unit 220 to store the heat flow sensor signal as the heat balance information.

The controller 210 in the information processing apparatus 200 then calculates the comfort level of the user 800 with reference to the first reference information (Activity A220). To rephrase this activity into a step, in the calculation step, the comfort level of the user 800 is calculated based on the heat balance information of the user 800 and the first reference information. Here, the first reference information is information indicating a relationship between the heat balance information of a user and the comfort level of the user. The first reference information may be a trained model that has learned the relationship between the heat balance information of a user and the comfort level of the user. According to an aspect that refers to the trained model, the evaluation of the comfort level can be optimized for each individual.

In Activity A220, for example, the following three-step information processing is executed. (1) The controller 210 reads the heat balance information from the storage unit 220. (2) The controller 210 refers to the first reference information and executes calculation processing. (3) The controller 210 causes the storage unit 220 to store the calculated comfort level of the user 800.

The controller 210 in the information processing apparatus 200 then calculates core temperature of the user 800 by referring to a predetermined calculation formula (Activity A230). The predetermined calculation formula is not particularly limited as long as it can calculate the core temperature of the user 800 using the heat flow of the user 800. For example, the predetermined calculation formula may be a formula using the heat flow of the user 800 and the heat resistance from the core part of the user 800 to the heat flow sensor 500. To rephrase this activity into a step, in the calculation step, the core temperature of the user 800 is further calculated based on the heat flow of the user 800 and the predetermined calculation formula.

In Activity A230, for example, the following three-step information processing is executed. (1) The controller 210 reads the heat flow of the user 800 from the storage unit 220. (2) The controller 210 refers to the predetermined calculation formula and executes the calculation processing. (3) The controller 210 causes the storage unit 220 to store the calculated core temperature of the user 800.

The controller 210 in the information processing apparatus 200 then calculates the risk of heatstroke for the user 800 with reference to a second reference information (Activity A240). The risk of heatstroke may be indicated as a thermal index, and may be, for example, wet bulb globe temperature (WBGT). To rephrase this activity into a step, in the calculation step, the risk of heatstroke for the user 800 is further calculated based on the core temperature of the user 800 and the second reference information. Here, the risk of heatstroke is included in the information related to the comfort level of the user 800. The second reference information is information indicating a relationship between the core temperature of a user and the risk of heatstroke for the user. According to such an aspect, it is possible to inform each individual of the risk of heatstroke.

In Activity A240, for example, the following three-step information processing are executed. (1) The controller 210 reads the core temperature of the user 800 from the storage unit 220. (2) The controller 210 refers to the second reference information and executes calculation processing. (3) The controller 210 causes the storage unit 220 to store the calculated risk of heatstroke for the user 800.

Then, the controller 210 in the information processing apparatus 200 generates display information to be displayed on the smartphone 300 and a control signal for switching the operating state of the air conditioner 400 (Activity A250). The display information includes the comfort level of the user 800 and the risk of heatstroke for the user 800. The control signal is a signal for switching the operating state such as an operation mode, set temperature, set air volume, etc., and may be, for example, an infrared signal. For example, when the temperature is 35° C. and the user 800 feels hot and uncomfortable, i.e., the comfort level of the user 800 is low, a control signal for lowering the set temperature of the air conditioner 400 is generated.

In Activity A250, for example, the following four-step information processing is executed. (1) The controller 210 reads the risk of heatstroke for the user 800 from the storage unit 220. (2) The controller 210 reads the comfort level of the user 800 from the storage unit 220. (3) The controller 210 executes generation processing and generates display information and a control signal. (4) The controller 210 causes the storage unit 220 to store the generated display information and the control signal.

The controller 210 in the information processing apparatus 200 then transmits the display information to the smartphone 300 and transmits the control signal to the air conditioner 400 (Activity A260). To rephrase this activity into a step, in the output step, the information related to the comfort level of the user 800 is output. Here, the information related to the comfort level of the user 800 includes a control signal to the air conditioner 400 (air conditioning device). According to such an aspect, it is possible to construct an optimal space for each individual.

The information related to the comfort level of the user 800 also includes display information that displays an index of the comfort level of the user 800. The index of the comfort level may be displayed, for example, using a numerical value. Taking a temperature-humidity index as an example, a comfort standard may be set at 65 to 70, and in the case where the user 800 feels cold, a lower numeral value may be displayed according to the coldness, and in the case where the user 800 feels hot, a higher numeral value may be displayed according to the hotness. According to such an aspect, it is possible to provide feedback of the evaluation of the comfort level to each individual.

In Activity A260, for example, the following three-step information processing are executed. (1) The controller 210 reads display information and a control signal from the storage unit 220. (2) The controller 210 executes output processing. (3) The communication unit 250 transmits the display information to the smartphone 300 and transmits the control signal to the air conditioner 400.

The controller 310 in the smartphone 300 then receives the display information transmitted from the information processing apparatus 200 (Activity A270). In Activity A270, for example, the following two-step information processing are executed. (1) The communication unit 350 receives display information transmitted from the information processing apparatus 200. (2) The controller 310 causes the storage unit 320 to store the received display information.

The controller 310 in the smartphone 300 then displays, on the display unit 330, the comfort level of the user 800 and the risk of heatstroke for the user 800 included in the display information (Activity A280). In Activity A280, for example, the following three-step information processing are executed. (1) The controller 310 reads display information from the storage unit 320. (2) The controller 310 executes display processing. (3) The display unit 330 displays the comfort level of the user 800 and the risk of heatstroke for the user 800.

Following Activity A260, the controller 410 in the air conditioner 400 receives a control signal transmitted from the information processing apparatus 200 (Activity A290). In Activity A290, for example, the following two-step information processing are executed. (1) The communication unit 450 receives a control signal transmitted from the information processing apparatus 200. (2) The controller 410 causes the storage unit 420 to store the received control signal.

The controller 410 in the air conditioner 400 then controls the operating state of the air conditioner 400 based on the received control signal (Activity A300). In the example described in Activity A250, the controller 410 controls the set temperature to be lowered by 1° C., for example.

Following Activity A260, the controller 210 in the information processing apparatus 200 proceeds to the processing of Activity A210 and repeatedly executes the processing of Activities A210 to A260. The processing by the controller 210 is repeated until the processing proceeds to Activity A140. In response to the processing by the controller 210, the controller 310 in the smartphone 300 executes processing of Activities A270 and A280, and the controller 410 in the air conditioner 400 executes processing of Activities A290 and A300.

According to the aspect of Section 4, it is possible to perform evaluation of the comfort level considering individual differences. In addition, due to a simple configuration, the saved resources can be used for other core functions.

### 5. Usage of Heat Flow Sensor 500

Section 5 describes usage of the heat flow sensor 500.

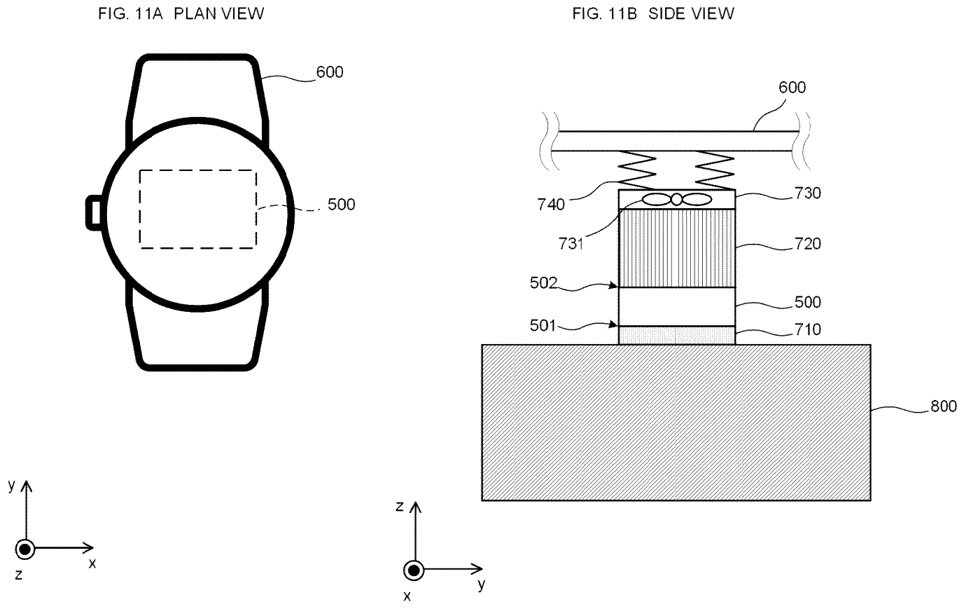

FIG. 11A and FIG. 11B are diagrams showing usage of the heat flow sensor 500 when the heat flow sensor 500 is attached to the skin of the user 800. FIG. 11A shows a plan view of an aspect in which the heat flow sensor 500 is attached. FIG. 11B shows a side view of the heat flow sensor 500 and each member attached to the heat flow sensor 500.

The heat flow sensor 500 may be attached to the skin (e.g., wrist) of the user 800 using a wrist watch type strap 600. In this case, the wrist watch type strap 600 presses the heat flow sensor 500 against the skin of the user 800. The wrist watch type strap 600 corresponds to a “biasing member” in the claims.

A gel sheet 710 (adhesive member) is attached to the heat flow sensor 500 to bond the heat flow sensor 500 to the skin of the user 800. The gel sheet 710 (adhesive member) is attached to the surface of the heat flow sensor 500 that faces the skin of the user 800. According to such an aspect, the heat flow can be stably acquired, and thus the accuracy of acquiring the heat flow can be improved.

A heat sink 720 is attached to the heat flow sensor 500. The heat sink 720 is attached to at least one of a front surface 501 (first surface) and a back surface 502 (second surface) of the heat flow sensor 500. Here, the front surface 501 is the surface from which the heat flow of the user 800 flows in, and the back surface 502 is the surface from which the heat flow of the user 800 flows out. In FIG. 11B, the heat sink 720 is attached to the front surface 501 of the heat flow sensor 500. According to such an aspect, the heat accumulated in the heat flow sensor 500 can be dissipated efficiently.

A fan case 730 including a fan 731 is attached to the heat flow sensor 500. The fan 731 has function of efficiently dissipating heat accumulated in the heat sink 720. The temperature of the back surface 502 (second surface) of the heat flow sensor 500 is controlled to be equal to the air temperature. According to such an aspect, excessive heat accumulation in the heat flow sensor 500 can be prevented, thereby improving the accuracy of acquiring the heat flow of the user 800.

A plate spring 740 (biasing member) is attached to the heat flow sensor 500. A plate spring 740 (biasing member) is attached so as to press the heat flow sensor 500 against the skin of the user 800. According to such an aspect, the heat flow can be stably acquired, and thus the accuracy of acquiring the heat flow can be improved.

Although the embodiments of the present disclosure have been described above, the present disclosure is not limited thereto and can be modified as appropriate without departing from the technical concept of the present disclosure.

### 6. Modified Example

Section 6 describes a modified example of the present embodiment.

An aspect of the present embodiment may be a program. This program causes a computer to execute each step of the information processing method of the present embodiment.

An aspect of the present embodiment may be a storage medium. This storage medium is a non-transitory computer readable storage medium. This storage medium stores a program that causes a computer to execute each step of the information processing method of the present embodiment.

The controller 210 executes a writing (memory) process and a reading process on various types of data and various types of information to and from the storage unit 220, but processes are not limited thereto, and the controller 210 may use, for example, a register or a cache memory in the controller 210 to execute information processing for each activity.

The first reference information may be information indicating a relationship between the heat balance information of a user and the comfort level of the user. Also, the first reference information may be a database that stores look-up tables, etc., a function that represents correspondence of value determined depending on certain variable, a mathematical model that mathematically relates a plurality of pieces of information, or the like.

The second reference information may be information indicating a relationship between the core temperature of a user and the risk of heatstroke for the user. Also, the second reference information may be a database that stores look-up tables, etc., a function that represents correspondence of values determined depending on certain variable, a mathematical model that mathematically relates a plurality of pieces of information, a trained model that has learned the relationships between a plurality of pieces of information, or the like.

The third reference information may be information indicating a relationship between the heat flow of a user and the comfort level of the user. Also, the third reference information may be a database that stores look-up table, etc., a function that represents correspondence of values determined depending on certain variable, a mathematical model that mathematically relates a plurality of pieces of information, a trained model that has learned the relationships between a plurality of pieces of information, or the like.

The heat balance information may further include at least one of air temperature, humidity, the body temperature of the user 800, air pressure, the core temperature of the user 800, wind speed, the activity of the user 800, the posture of the user 800, and the action history of the user 800. By further using such information, the accuracy of the comfort level evaluation can be improved.

The biasing member is not limited to the wrist watch type strap 600 and the plate spring 740, such as a coil spring, as long as it is attached so as to press the heat flow sensor 500 against the skin of the user 800.

The adhesive member is not limited to the gel sheet 710, such as a tape-like adhesive sheet, as long as it can bond the skin of the user 800 to the heat flow sensor 500.

### 7. Others

The present disclosure may be provided in each of the following aspects.

(1) An information processing method, comprising: an acquisition step of acquiring heat balance information that is information related to heat balance of a user, the heat balance information including a heat flow of the user; a calculation step of calculating a comfort level of the user based on the heat balance information and a first reference information that is information indicating a relationship between the heat balance information and the comfort level; and an output step of outputting information related to the comfort level.

According to this approach, it is possible to perform evaluation of the comfort level considering individual differences. In addition, due to a simple configuration, the saved resources can be used for other core functions.

(2) The information processing method according to (1), wherein: the information related to the comfort level includes a control signal to an air conditioning device.

According to such an aspect, it is possible to construct an optimal space for each individual.

(3) The information processing method according to (1) or (2), wherein: the information related to the comfort level includes display information that displays an index of the comfort level.

According to such an aspect, it is possible to provide feedback of the evaluation of the comfort level to each individual.

(4) The information processing method according to any one of (1) to (3), wherein: the calculation step further includes steps of: further calculating core temperature of the user based on the heat flow and a predetermined calculation formula, and further calculating a risk of heatstroke for the user based on the core temperature and a second reference information, wherein the risk of heatstroke is included in the information related to the comfort level, and the second reference information is information indicating a relationship between the core temperature and the risk of heatstroke.

According to such an aspect, it is possible to inform each individual of the risk of heatstroke.

(5) The information processing method according to any one of (1) to (4), wherein: the heat balance information further includes at least one of air temperature, humidity, body temperature of the user, air pressure, core temperature of the user, wind speed, activity of the user, posture of the user, and an action history of the user.

According to such an aspect, the accuracy of the comfort level evaluation can be improved.

(6) The information processing method according to any one of (1) to (5), wherein: the first reference information is a trained model that has learned the relationship between the heat balance information and the comfort level.

According to such an aspect, the evaluation of the comfort level can be optimized for each individual.

(7) A non-transitory computer readable storage medium, storing a program configured to cause the computer to execute each step of the information processing method according to any one of (1) to (6).

According to this approach, it is possible to perform evaluation of the comfort level considering individual differences. In addition, due to a simple configuration, the saved resources can be used for other core functions.

(8) A calculation method for calculating a comfort level of a user by using a heat flow sensor, the method comprising: an arrangement step of arranging the heat flow sensor at a position where a heat flow of the user is measurable; a measurement step of measuring the heat flow by using the heat flow sensor; and a calculation step of calculating the comfort level based on the heat flow and a third reference information that is information indicating a relationship between the heat flow and the comfort level.

According to this approach, it is possible to perform evaluation of the comfort level considering individual differences. In addition, due to a simple configuration, the saved resources can be used for other core functions.

(9) The calculation method according to (8), wherein: the arrangement step includes attaching the heat flow sensor to the user.

According to such an aspect, the heat flow of the user can be acquired more reliably.

(10) The calculation method according to (8) or (9), wherein: the arrangement step includes attaching the heat flow sensor to a skin of the user.

According to such an aspect, the heat flow of the user can be acquired more further reliably.

(11) The calculation method according to any one of (8) to (10), wherein: the arrangement step includes arranging the heat flow sensor on a seat surface on which the user sits.

According to such an aspect, the heat flow of the user can be acquired more reliably.

(12) The calculation method according to any one of (8) to (11), wherein: a heat sink is attached to the heat flow sensor, and the heat sink is attached to at least one of a first surface and a second surface of the heat flow sensor.

According to such an aspect, the heat accumulated in the heat flow sensor can be dissipated efficiently.

(13) The calculation method according to any one of (8) to (12), wherein: a biasing member is attached to the heat flow sensor, and the biasing member is attached so as to press the heat flow sensor against a skin of the user.

According to such an aspect, the accuracy of acquiring the heat flow can be improved.

(14) The calculation method according to any one of (8) to (13), wherein: an adhesive member is attached to the heat flow sensor to bond a skin of the user and the heat flow sensor, and the adhesive member is attached to a surface of the heat flow sensor that faces the skin of the user.

According to such an aspect, the accuracy of acquiring the heat flow can be improved.

(15) The calculation method according to any one of (8) to (14), wherein: temperature of a second surface of the heat flow sensor is controlled to be equal to air temperature, and the second surface is a surface from which the heat flow flows out.

According to such an aspect, the accuracy of acquiring the heat flow can be improved.

(16) The calculation method according to any one of (8) to (15), wherein: the arrangement step includes arranging a plurality of the heat flow sensors at positions where the heat flow is measurable, and the measurement steps includes measuring the heat flow from each of the heat flow sensors.

According to such an aspect, the influence of thermal resistance in the heat flow sensor can be reduced.

Of course, the present disclosure is not limited to the above aspects.

1. An information processing method, comprising:
an acquisition step of acquiring heat balance information related to heat balance of a user, the heat balance information including a heat flow information of the user;
a calculation step of calculating a comfort level of the user based on the heat balance information and a first reference information indicating a relationship between the heat balance information and the comfort level; and
an output step of outputting information related to the comfort level.

2. The information processing method according to claim 1, wherein:
the information related to the comfort level includes a control signal for controlling an air conditioning device.

3. The information processing method according to claim 1, wherein:
the information related to the comfort level includes display information for displaying an index of the comfort level.

4. The information processing method according to claim 1, wherein:
the calculation step further includes steps of:
further calculating core temperature of the user based on the heat flow and a predetermined calculation formula, and
further calculating a risk of heatstroke for the user based on the core temperature and a second reference information,
wherein the risk of heatstroke is included in the information related to the comfort level, and
the second reference information is information indicating a relationship between the core temperature and the risk of heatstroke.

5. The information processing method according to claim 1, wherein:
the heat balance information further includes at least one of air temperature, humidity, body temperature of the user, air pressure, core temperature of the user, wind speed, activity of the user, posture of the user, and an action history of the user.

6. The information processing method according to claim 1, wherein:
the first reference information is a trained model obtained by training on the relationship between the heat balance information and the comfort level.

7. A non-transitory computer readable storage medium,
storing a program configured to cause the computer to execute each step of the information processing method according to claim 1.

8. A calculation method for calculating a comfort level of a user by using a heat flow sensor, the method comprising:
an arrangement step of placing the heat flow sensor at a position where a heat flow of the user is measurable;
a measurement step of measuring the heat flow by using the heat flow sensor; and
a calculation step of calculating the comfort level based on the heat flow and a third reference information that is information indicating a relationship between the heat flow and the comfort level.

9. The calculation method according to claim 8, wherein:
the arrangement step includes attaching the heat flow sensor to the user.

10. The calculation method according to claim 8, wherein:
the arrangement step includes attaching the heat flow sensor to a skin of the user.

11. The calculation method according to claim 8, wherein:
the arrangement step includes arranging the heat flow sensor on a seat surface on which the user sits.

12. The calculation method according to claim 8, wherein:
a heat sink is attached to the heat flow sensor, and
the heat sink is attached to at least one of a first surface and a second surface of the heat flow sensor.

13. The calculation method according to claim 8, wherein:
a biasing member is attached to the heat flow sensor, so as to press the heat flow sensor against a skin of the user.

14. The calculation method according to claim 8, wherein:
an adhesive member is attached to the heat flow sensor to bond a skin of the user and the heat flow sensor, and
the adhesive member is attached to a surface of the heat flow sensor that faces the skin of the user.

15. The calculation method according to claim 8, wherein:
temperature of a second surface of the heat flow sensor is controlled to be equal to air temperature, and
the second surface is a surface from which the heat flow flows out.

16. The calculation method according to claim 8, wherein:
the arrangement step includes arranging a plurality of the heat flow sensors at positions where the heat flow is measurable, and
the measurement step includes measuring the heat flow from each of the heat flow sensors.
